(19) United States
(12) Patent Application Publication
(10) Pub. No.: US 2020/0074625 A1
(43) Pub. Date: Mar. 5, 2020

(54) FUNCTIONAL MEASUREMENTS IN ECHOCARDIOGRAPHY

(71) Applicant: Norwegian University of Science and Technology, Trondheim (NO)

(72) Inventors: Andreas ØSTVIK, Trondheim (NO); Erik SMISTAD, Trondheim (NO); Lasse LØVSTAKKEN, Trondheim (NO)

(21) Appl. No.: 16/559,931

(22) Filed: Sep. 4, 2019

Related U.S. Application Data
(60) Provisional application No. 62/727,075, filed on Sep. 5, 2018.

(51) Int. Cl. G06T 7/00; G06N 3/04; G16H 30/40; G06T 7/11; G06T 7/20; G06K 9/62; A61B 8/08; A61B 8/14

(52) U.S. Cl. CPC G06T 7/0012; G06T 2200/24; G16H 30/40; G06T 7/11; G06T 7/20; G06K 9/6267; G06K 9/6261; G06K 9/6228; A61B 8/0883; A61B 8/14; G06T 2207/10132; G06T 2207/30048; G06T 2207/20084; G06K 2209/051; G06N 3/0454

(57) ABSTRACT

A method for processing echocardiography data to enable automatic functional measurements based on cardiac ultrasound images as an input, including (i) classification of the cardiac ultrasound images to ensure that relevant images are passed on to the next steps, optionally utilising a first neural network, such as a convolutional neural network, (ii) segmentation and semantic partitioning of the left ventricle (LV) myocardium to extract relevant parts of the image, optionally by using a second neural network, (iii) regional motion estimates to determine a mapping of displacements in the extracted parts of the image and to output estimated tissue motion vectors for the extracted parts of the image, optionally using a third neural network, and (iv) fusion of measurements via state estimation applied to the tissue motion vectors and thereby incorporating a temporal domain to produce data showing variation of the estimated measurements over time.

### TECHNICAL FIELD

The invention relates to a method for processing echocardiography data in order to allow for functional measurements to be obtained automatically. The invention further provides corresponding computer programme products and systems.

### BACKGROUND OF THE INVENTION

Recent years have shown that quantitative assessment of cardiac function has become indispensable in echocardiography. Evaluation of the hearts contractile apparatus has traditionally been limited to geometric measures such as ejection fraction (EF) and visual estimation (eyeballing) of myocardial morphophysiology. Despite being a central part of standard protocol examinations at the outpatient clinic, the methods tend to have poor inter- and intravariability. With tissue doppler imaging (TDI) and speckle tracking (ST), the quantification tools have moved beyond these measures, and enabled new methods for assessing the myocardial deformation pattern. Myocardial deformation imaging, e.g. strain and strain rate, derived from TDI and ST, have high sensitivity, and can allow an earlier detection of cardiac dysfunction. However, these methods also have several limitations. For instance, TDI is dependent on insonation angle, i.e. measurements are along the ultrasound beam. A poor parallel alignment with the myocardium can thus influence the results. Speckle tracking is less angle dependant (typically dependant on the lateral resolution), but has suffered from poor temporal resolution and ad-hoc setups.

### SUMMARY OF THE INVENTION

Viewed from a first aspect, the invention provides a method for processing echocardiography data in order to enable automatic functional measurements based on cardiac ultrasound images as an input, the method comprising:

(i) classification of the cardiac ultrasound images in order to ensure that relevant images are passed on to the next steps;

(ii) segmentation and semantic partitioning of the left ventricle (LV) myocardium to extract relevant parts of the image;

(iii) regional motion estimates to determine a mapping of displacements in the extracted parts of the image and to output estimated tissue motion vectors for the extracted parts of the image; and

(iv) fusion of measurements via state estimation applied to the tissue motion vectors and thereby incorporating a temporal domain to produce data showing variation of the estimated measurements over time;

wherein at least one of steps (i), (ii) and (iii) is done using a neural network, or a part of a neural network.

With this method motion estimation from deep learning is applied in echocardiography in order to allow for a fully automatic approach, without the need for any manual input, such as for identifying the relevant areas via masks and so on. Automatic view classification and segmentation of relevant cardiac structures in echocardiography has been a topic of great interest and research but to the inventors' knowledge, no published study has utilized the same techniques in order to provide a fully automatic approach using at least one neural network, or at least one part of a neural network and hence taking advantage of the benefits of “deep learning”. These methods can be more robust in terms of noise and small displacements than traditional optical flow methods, thus they are appealing for ultrasound and myocardial motion estimation. The method above allows for fast and fully automated pipelines for calculating clinically relevant parameters, with feasibility of on-site analysis such as by enabling automatic calculation of strain (such as the global longitudinal strain (GLS)) from cardiac views, such as the four-chamber view, in an end-to-end fashion. In the example where GLS is estimated then the method may comprise automated recognition of the apical views within the cardiac views.

The method of the first aspect may be performed via multiple neural networks or via a single neural network with parts (such as the first and second part referenced below) having sub-functions associated with the different steps of the proposed method. Thus, step (i) may be done by utilising a first neural network, or first part of a neural network, such as a convolutional neural network. Alternatively or additionally, step (ii) may be done by using a second neural network, or second part of a neural network. Alternatively or additionally, step (iii) may be done by using a third neural network, or a third part of a neural network. The invention extends to any one of steps (i), (ii) and (iii) being performed using the respective neural network, or part of a neural network, as well as to any two of the three steps being performed using the respective neural network, or part of a neural network. In one example all three of steps (i), (ii) and (iii) are performed using the respective neural network, or part of a neural network. The method may use a neural network with three parts for providing the respective parts for steps (i), (ii) and (iii).

In some examples, step (i) uses a convolutional neural network (CNN) in the form of a feed-forward CNN composed of inception blocks and a dense connectivity pattern. In one example, input is propagated through two component blocks with (3×3) convolution kernels followed by max pooling. The first and second convolution layer may have 16 and 32 filters respectively. The pooling may have size (2×2) and equal strides. After the second pooling layer, data may be processed through an inception module with three parallel routes. In the example embodiment, each route consists of a bottleneck, two of which may be followed by blocks with larger convolution kernels, i.e. (3×3) and (5×5) respectively. The input of the inception module may be concatenated with the output and processed into a transition module with bottleneck and max pooling. In the example embodiment, this step is repeated three times, and we double the amount of filters before every new pooling layer. After the third transition, the data may be processed through two inception blocks with constant amount of filters and no pooling. The route with (5×5) convolution kernels may be omitted in these modules, with dropout regularization being used between them. The final classification block optionally consists of a compressing convolution layer with (1×1) kernels and number of filters equal to the class count. This may be activated with another PReLU, before features are spatially averaged and fed into a softmax activation.

In the example embodiment, training may be performed from scratch with Adam optimizer and categorical cross entropy loss, with input size of (128×128) greyscale. A total of eight classes may be used for training: the apical four chamber, two chamber and long-axis, the parasternal long- and short-axis, subcostal four-chamber and vena cava inferior, as well as a class for unknown data. With this example for step (i) the final network classifies the different cardiac views, and if applicable, i.e. high confidence of apical four-chamber, the image is processed into the remaining processing chain.

In step (ii) the neural net (or part thereof) may also classify the left ventrical myocardium. Optionally, the extracted parts of the image may be masked areas extracted using masking.

The segmentation at step (ii) may use a U-Net type of CNN classify the LV myocardium with the pixel map of the segmentation being processed and used to define regions of interest (ROI) and the basis of measurement kernels. The segments may be used to regularize the motion estimates by removing noise outside the tissue. The motion may then be densely estimated frame-to-frame in the domain of interest. The architecture of an example CNN for this step consists of a downsampling, and an upsampling part of five levels with concatenating cross-over connection between equally sized feature maps. Each level may have two convolution layers with the same amount of filters ranging from 32 to 128 from top to bottom respectively. In the example all filters have a size of (3×3), Max pooling with size (2×2) and equal strides was used for downsampling and nearest neighbour for upsampling. Training may be performed with Adam optimizer and Dice loss.

The segmentation is optionally used a basis for two different tasks, including the masking of the first aspect and also centerline extraction. In an example the contour of the segmentation mask is used to define the endo- and epicardial borders, and further the centerline C={(x,y)₁, . . . , (x,y)_{N}} may be sampled along the myocard, for example there may be sampling with N=120 equally spaced points. The latter may be passed to the Kalman filter at step (iv).

At step (iii) motion estimation is carried out. In one example this uses the neural networks referred to as FlowNets. The preferred design involves stacking of multiple U-Net architectures with image warping of intermediate motion and propagation of brightness error. The output prediction of the network may be dense tissue motion vectors in the masked area.

In the example embodiment two parallel routes are created for the motion estimation in order to tackle large and small displacements separately. The large displacements may be predicted by stacking three U-Net architectures, the first which includes explicit correlation of feature maps, while the two succeeding are standard U-Net architectures without custom layers. For small displacements, in the preferred implementation only one U-Net is used, but compared to the networks for large displacements, the kernel size and stride of the first layer is reduced. With this example then the outputs from two routes are fused together at the end with a simple CNN. The networks may be trained separately, for example by using a schedule consisting of different synthetic datasets with a wide range of motion vector representations. The small displacement network may be fine-tuned on a dataset modified for subpixel motion. Adam optimizer and endpoint error loss may be used while training for all the networks. In this example the input size of the network was kept the same as the original implementation, i.e. (512×384).

At step (iv) measurements are fused via state estimation (for example via a Kalman filter) employed on every point-velocity component in the measurement kernel (i.e. all data from the masked region) with a constant acceleration model. Advantages of this step include temporal smoothing, reducing potential burst noise detectable in image-to-image measurements.

Subsequent to steps (i) to (iv) the output estimations may be used in the calculation of various clinical indices, and hence in some examples the method includes a further step (v), which comprises calculation of clinical indices.

In an example step (v) uses the updated point-velocity components from step (iv) in order to calculate one or more clinical indices, such as one or more of:
Global and regional longitudinal myocardial strain
Global and regional longitudinal myocardial strain rate
Regional myocardial velocity
Regional myocardial displacement

The method may comprise accessing cardiac B-mode ultrasound data as the input data; processing the ultrasound data through steps (i) to (iv) and subsequently providing regional numeric deformation values for all segments of the left ventricle.

In some examples, cardiac cycles which have inferior quality for robust deformation estimation are automatically discarded during initial processing at step (i).

The automatic discarding of ultrasound data may occur when view recognition fails due to view recognition confidence being lower than a threshold. Optionally the view recognition confidence may be calculated by the percentage of image frames classified to the same view compared to all frames classified from that cycle.

The automatic discarding of ultrasound data may occur when segmentation indicates that apex is moving away and towards the probe during the cardiac cycle or when regularization is failing.

The method may be applied to multiple cardiac cycles, including cycles obtained in real-time and/or stored cardiac cycles. It may be applied to several stored cardiac cycles from one patient or to several stored cardiac cycles from different patients.

Results from several stored cardiac cycles from different patients may be used to compare regional strain between patient groups.

In some examples the method outputs information via a user interface. This may display a trend graph for regional and/or global values based on processing of current and prior exams for the same patient.

In one example embodiment, at the end of an examination, the user is automatically presented with a Left ventricle segmental bullseye generated in background based on processing recorded images while the user was completing the exam. Thus, processing may advantageously occur automatically on apical B-mode images while the user continues to record colorflow and Doppler images.

The processing of images via any or all of steps (i) to (iv) and optionally (v) may occur in the background while the user continues to record images. In that case a warning may be presented if it is determined that measurements show a possible problem. For example, if a left ventricle segment falls outside normal deformation value ranges, then a warning highlighting this segment and its non-normal value may be presented to the user.

In another aspect, the invention provides a computer programme product containing instructions that, when executed, will configure a computer device to carry out the method of the first aspect, optionally including other features of the method as discussed above.

Thus, the computer programme product may contain instructions that, when executed, will configure the computer device to process cardiac ultrasound images via a method comprising:

(i) classification of the cardiac ultrasound images utilising a first neural network such as a convolutional neural network in order to ensure that relevant images are passed on to the next steps;

(ii) segmentation and semantic partitioning of the left ventricle (LV) myocardium by using a second neural network to classify the left ventrical myocardium and use masks to extract relevant parts of the image;

(iii) regional motion estimates using a third neural network to determine a mapping of displacements in the masked areas and to output estimated tissue motion vectors for the masked areas; and

(iv) fusion of measurements via a Kalman filter applied to the tissue motion vectors and thereby incorporating a temporal domain to produce data showing variation of the estimated measurements over time;

and optionally any of the other features of the method(s) set forth above.

In yet another aspect, the invention provides a computer device for carrying out the method of the first aspect. This may be a computer device configured to perform the method, optionally being so configured via use of a computer programme product as discussed above. The computer device may be a part of an ultrasound imaging system and hence the invention further extends to an ultrasound imaging system comprising a computer device for carrying out the method of the first aspect. In this case the computer device is used for processing ultrasound images obtained via the imaging system and may form a part of a control system of the ultrasound imaging system.

The computer device may hence be configured to process cardiac ultrasound images via a method comprising:

(i) classification of the cardiac ultrasound images utilising a first neural network such as a convolutional neural network in order to ensure that relevant images are passed on to the next steps;

(ii) segmentation and semantic partitioning of the left ventricle (LV) myocardium by using a second neural network to classify the left ventrical myocardium and use masks to extract relevant parts of the image;

(iii) regional motion estimates using a third neural network to determine a mapping of displacements in the masked areas and to output estimated tissue motion vectors for the masked areas; and

(iv) fusion of measurements via a Kalman filter applied to the tissue motion vectors and thereby incorporating a temporal domain to produce data showing variation of the estimated measurements over time;

and optionally any of the other features of the method(s) set forth above.

### BRIEF DESCRIPTION OF THE DRAWINGS

An example embodiment of the invention will now be described by way of example only and with reference to the accompanying drawings, in which:

FIG. 1 shows a visualisation of a proposed measurement pipeline;

FIG. 2 shows detail of different steps of the pipeline of FIG. 1; and

FIG. 3 includes plots of the results of an assessment of global longitudinal strain (GLS) used to evaluate the performance of the pipeline of FIG. 1.

### DETAILED DESCRIPTION

As described in further detail below, a method has been developed that enables automatic functional measurements in 2D echocardiography. The system works in an end-to-end fashion with standard cardiac ultrasound images as input, and several clinical measures, as well as motion estimates and regional masks as direct output.

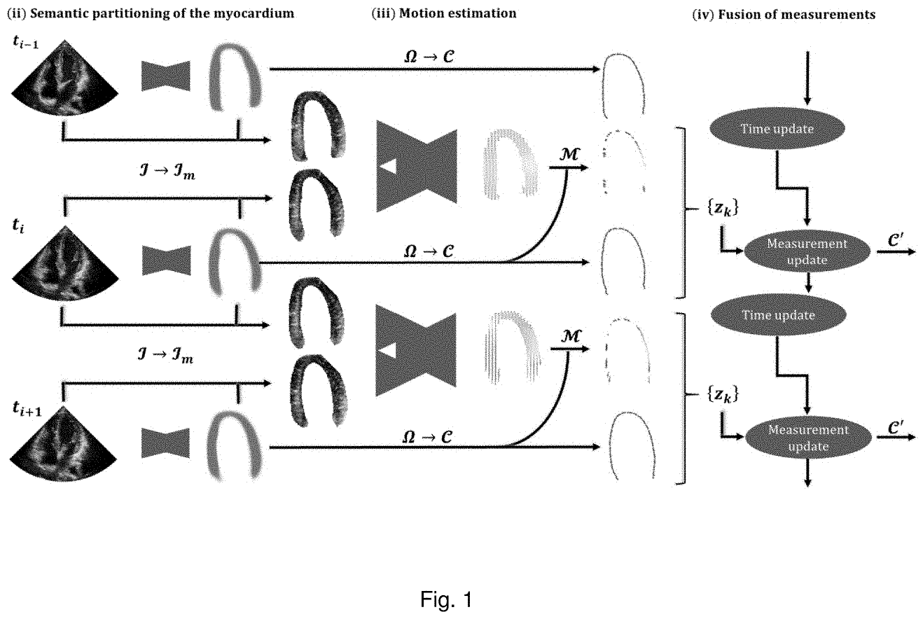

The method core is comprised of five components, (i) classification of cardiac view, (ii) segmentation and semantic partitioning of the left ventricle (LV) myocardium, (iii) regional motion estimates, (iv) fusion of measurements and (v) calculation of clinical indices. An illustration of an example setup for measuring global longitudinal strain after view classification is illustrated in FIG. 1. By way of an overview, FIG. 1 shows how ultrasound images are forwarded through a segmentation network, and the resulting masks are used to extract relevant parts of the image. The masked ultrasound data is further processed through the motion estimation network yielding a map of velocities. Here, the centerline position and velocities of the myocard are used in the measurement update step of a Kalman filter. Note that the measurement kernels can be set arbitrary based on the segmentation. The updated results are used as a basis for estimating clinical indices such as global or regional longitudinal strain.

The steps (i) to (iv) summarised above are implemented as follows:

(i) Cardiac View Classification:

Utilizes a convolutional neural network (CNN) to classify cardiac views. This is used to assure that relevant images obtained in the apical acoustic window are forwarded through the measurement pipeline.

The view classification is the first essential step in the automatic pipeline, and is used to quality assure and sort incoming data. We employ a feed-forward CNN composed of inception blocks and a dense connectivity pattern. Initially, input is propagated through two component blocks with (3×3) convolution kernels followed by max pooling. The first and second convolution layer has 16 and 32 filters respectively. We use pooling with size (2×2) and equal strides. After the second pooling layer, data is processed through an inception module with three parallel routes. Each route consist of a bottleneck, two of which were followed by blocks with larger convolution kernels, i.e. (3×3) and (5×5) respectively. The input of the inception module is concatenated with the output and processed into a transition module with bottleneck and max pooling. This step is repeated three times, and we double the amount of filters before every new pooling layer. The dense connectivity pattern alleviates the vanishing gradient problem, and can enhance feature propagation and reusability. After the third transition, the data is processed through two inception blocks with constant amount of filters and no pooling. The route with (5×5) convolution kernels is omitted in these modules, and dropout regularization was used between them. The final classification block consists of a compressing convolution layer with (1×1) kernels and number of filters equal to the class count. This is activated with another PReLU, before features are spatially averaged and fed into a softmax activation.

Training is performed from scratch with Adam optimizer and categorical cross entropy loss, with input size of (128×128) greyscale. A total of eight classes were used for training, the apical four chamber, two chamber and long-axis, the parasternal long- and short-axis, subcostal four-chamber and vena cava inferior, as well as a class for unknown data. The final network classifies the different cardiac views, and if applicable, i.e. high confidence of apical four-chamber, the image is processed into the remaining processing chain.

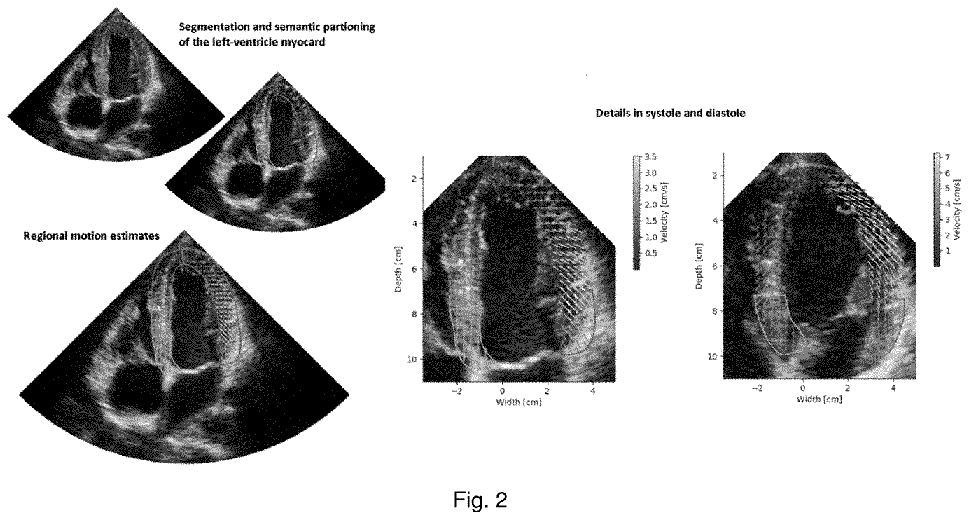

(ii) Segmentation: Utilizes a U-Net type of CNN classify the LV myocardium. The pixel map of the segmentation is processed and used to define regions of interest (ROI) and the basis of measurement kernels. The implementation allows high modularity within the segmentation masks in terms of measurements kernels, e.g. regional (base, mid, apical), centerlines with different widths, points, epicard and endocard borderlines. See FIG. 2 for an example of the segmentation and regional measurement kernels. FIG. 1 shows an example with a centerline measurement kernel. In FIG. 2 the top left corner illustrates the segmentation and partitioning of the left-ventricle myocard in the apical four-chamber view. The segments are used to regularize the motion estimates by removing noise outside the tissue. The motion is then densely estimated frame-to-frame in the domain of interest. Some details of the motion vectors at arbitrary stages of the cardiac cycle is shown to the right. As is prevalent, the methods achieve expected performance in the valid range of motion and are not dependent on insonation angle.

A standard U-Net type of CNN is utilized. The architecture consists of a downsampling, and an upsampling part of five levels with concatenating cross-over connection between equally sized feature maps. Each level has two convolution layers with the same amount of filters ranging from 32 to 128 from top to bottom respectively. All filters have a size of (3×3), Max pooling with size (2×2) and equal strides was used for downsampling and nearest neighbour for upsampling. Training was performed with Adam optimizer and Dice loss, and the size of the input image was set to (256×256) greyscale. The output of the network is a segmentation mask Ω.

The segmentation is used a basis for two different tasks, masking the input of the motion estimation network Iₘ, and centerline extraction. We mask the ultrasound image I to remove redundant input signal. The contour of the segmentation Ω was used to define the endo- and epicardial borders, and further the centerline C=[(x,y)₁, . . . , (x,y)_{N}] was sampled between with N=120 equally spaced points along the myocard. The latter is passed to the Kalman filter.

Both networks are trained on data from up to 500 patients annotated by experts and the results are extensively evaluated with state-of-the-art accuracy. The two preceding steps hence include the following advances:
Real-time classification of standard views in transthoracic echocardiography using convolutional neural networks.
Automatic, fast and accurate cardiac ultrasound segmentation using deep learning.

(iii) Motion estimation: the motion estimation step has been developed based on the work done by Ilg et al in the paper entitled “Flownet 2.0: Evolution of optical flow estimation with deep networks”, IEEE conference on computer vision and pattern recognition (CVPR), Vol. 2, 2017. It thus uses the networks referred to as FlowNets. The design involves stacking of multiple U-Net architectures with image warping of intermediate motion and propagation of brightness error. The output prediction of the network is dense tissue motion vectors in the masked area.

Two parallel routes are created to tackle large and small displacements separately. The prior is solved by stacking three U-Net architectures, the first which includes explicit correlation of feature maps, while the two succeeding are standard U-Net architectures without custom layers. For small displacement, only one U-Net is used, but compared to the networks for large displacements, the kernel size and stride of the first layer is reduced. At the end, the two routes are fused together with a simple CNN. Proper handling of variable motion is essential in echocardiography, and such a complex setup can learn to reproduce motion from the whole cardiac cycle. The networks are trained separately, in a schedule consisting of different synthetic datasets with a wide range of motion vector representations. The small displacement network is fine-tuned on a dataset modified for subpixel motion. Adam optimizer and endpoint error loss is used while training for all the networks. In this example the input size of the network was kept the same as the original implementation, i.e. (512×384).

The output prediction of the network is dense tissue motion vectors in the masked ultrasound area. The C of the current segmentation is used to extract the corresponding set of motion vectors M={(vₓ,v_{y})₁, . . . , (vₓ,v_{y})_{N}}. Disregarding the fundamentals of motion, the datasets have no resemblance to echocardiography. We assume that more relevant data with ground truth information, such as simulations, can improve the results.

(iv) Fusion of estimations: Performed by employing a Kalman filter on every point-velocity component in the measurement kernel with a constant acceleration model. In addition to the motion estimation, this serves as a simple method for further incorporating the temporal domain, which is natural in the context of echocardiography. It adds temporal smoothing, reducing potential burst noise detectable in image-to-image measurements. The updated point-velocity components are then used to calculate several clinical indices. The updated centreline C′ is used to calculate the longitudinal ventricular length, t, i.e. the arc length, for each timestep t. Further, this is used to estimate the global longitudinal strain ϵ(t)=(ι(t)−ι₀)/ι₀ along the center of the myocard.

Subsequent to steps (i) to (iv) the output estimations may be used in the calculation of various clinical indices, and hence a further step may be:

(v) Calculation of clinical indices: The current implementation features calculations of the following indices of LV function:
Global and regional longitudinal myocardial strain
Global and regional longitudinal myocardial strain rate
Regional myocardial velocity
Regional myocardial displacement

Summarized, the method may provide one or more of the following:
A modular implementation and extensive details about the methods.
A setup for training the convolutional neural networks with own data or existing methods.
Extraction of specific components for use in other pipelines and methods (e.g. motion estimation).
Deployable models with trained parameters ready for use (current implementation supports Tensorflow backend).

The different CNNs used in this system are based on publicly available architectures and thus each are readily implemented with appropriate knowledge of the teaching presented herein. However, the pipeline composition and corresponding synergy together with traditional methods has not previously been attempted. The combination of features set out herein facilitates a simple and standardized approach to cardiac functional imaging enabling fully automated processing of ultrasound data making use of the advantages provided by deep learning techniques. Some potential key points for this innovation include:
Fusion of segmentation and motion estimation. Combining sensory data from disparate sources will potentially result in less uncertainty than the sources being used individually.
Regularization of motion measurements by removing signal outside the region of interest based on segmentation of the myocard.
The motion estimation network can learn highly non-linear motion, and can overcome the limitations and rigidity of model based methods.
The setup can learn to reproduce results of existing methods, such as speckle tracking or simulators.
The method is fully automatic and requires no interaction by the user other than supplying ultrasound data.

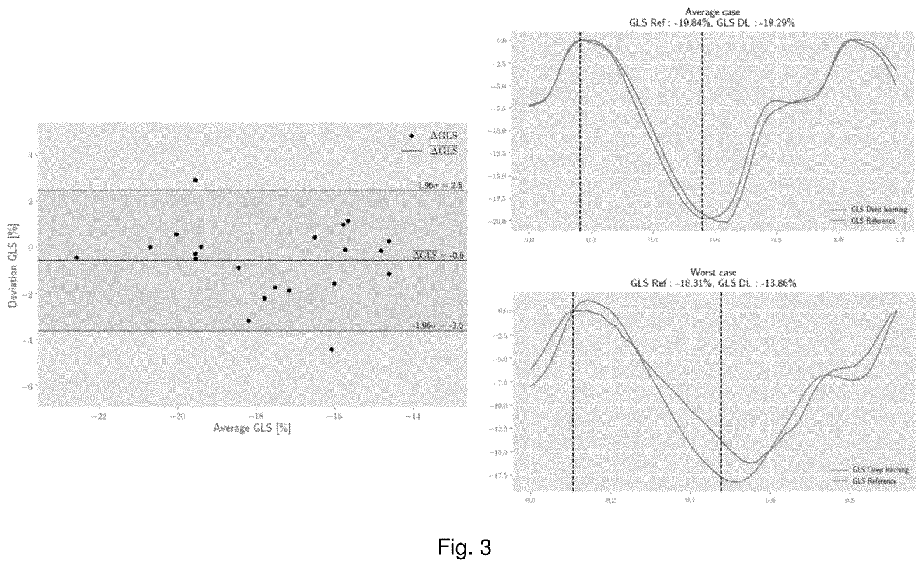

As an example of the effectiveness of the proposed method FIG. 3 shows the results of an assessment of global longitudinal strain (GLS) using the pipeline of FIG. 1 and the method described above. For the purposes of this example anonymous echocardiography data for training the view classification and segmentation models was acquired from various patient studies with Research Ethics Board (REB) approval. The echocardiography data used are obtained at various outpatient clinics with GE Vivid E9 or Vivid E95 ultrasound systems (GE Vingmed Ultrasound, Horten, Norway), and consist of data from over 250 patients. The health status of individual subjects is unknown, but it is representative for a standard outpatient clinic. The data includes manual expert annotation of views, and the epi- and endocard borders of the left ventricle. The view classification and segmentation networks are trained separately on this data, with a significant fraction left out for testing to ensure that the automatic identification of the epi- and endocard borders is accurate. The motion estimation network was trained on two synthetic dataset, namely FlyingChairs and FlyingThings3D. Disregarding the fundamentals of motion, the datasets have no resemblance to echocardiography. However, they can be modified to have representations covering both sub- and superpixel motion, which is necessary to reproduce motion from the whole cardiac cycle.

For validation of GLS, 21 subjects called for evaluation of cardiac disease in two clinical studies were included. Both are REB approved, and informed consent was given. Two specialists in cardiology performed standard strain measurements using a semi-automatic method implemented in GE EchoPAC. The method uses speckle tracking to estimate myocardial deformation, but the methodology is unknown. The results were used as a reference for evaluating the implemented pipeline.

GLS was obtained successfully in all patients. The results for apical four-chamber views are displayed in FIG. 3, together with the GLS curves of the average and worst case subjects. The average deviation of the systolic GLS between the two methods was (−0.6±1.6)%. The average strain on all subjects (−017.9±2.3)% and (−17.3±2.5)%, for a reference method and for the proposed automated method respectively.

The view classification achieved an image-wise F₁ score of 97.9% on four-chamber data of 260 patients, and the segmentation a dice score of (−0.87±0.03) on 50 patients, all unknown and independent from the training set. The system was implemented as a Tensorflow dataflow graph}, enabling easy deployment and optimized inference. Using a modern laptop with a Nvidia GTX 1070 GPU, the average inference time was estimated to 115±1 ms per frame, where flow prediction accounts for approximately 70% of the runtime.

Compared to the reference method, the measurements from the proposed pipeline were slightly underestimated. The reference method is not a gold standard for GLS and might not necessarily yield correct results for all cases. Speckle tracking can fail where noise hampers the echogenicity. We could identify poor tracking in the apical area due to noise for some subjects, and this would in turn result in larger strain. Further, the vendor comparison study shows that the commercial system used in this study on average overestimates the mean of all vendors by 1.7%. This in mind, we note that the results from the current implementations are in the expected range. For individual cases, the deformation have overlapping and synchronized trends, as is prevalent from FIG. 3.

The proposed pipeline involves several sources of error, especially the segmentation and motion networks being the fundamental building blocks of the measurements. Using the segmentation mask to remove redundant signal in the ultrasound image seems feasible and useful for removing some noise in the motion network. However, it is not essential when measuring the components of the centerline, as they are far from the borders of the myocard, where the effect is noticable.

Future developments and potential refinements to the method include the addition of multiple views, e.g. apical two- and long-axis, allowing average GLS. This is considered a more robust metric, less prone to regional noise. Also, fusion of models are currently naive, and we expect results to improve inducing models with more relevance to cardiac motion. The same holds for the motion estimation, i.e. the network could benefit from training on more relevant data. Further, we wish to do this for regional strain measurements. For clinical validation, we need to systematically include the subject condition and a larger test material.

Thus, it will be apparent from the above that the proposed method can provide the following advantages and/or may include the features of the following clauses as alternatives to or in addition to the features set out in the appended claims:
An ultrasound processing method comprising: accessing cardiac B-mode ultrasound data; processing the ultrasound data through a neural network (Deep Learning) pipeline; at end of processing, providing regional numeric deformation values for all segments of the left ventricle.

OR
An ultrasound imaging system comprising: accessing cardiac B-mode ultrasound data; processing the ultrasound data through a neural network (Deep Learning) pipeline; at end of processing, providing regional numeric deformation values for all segments of the left ventricle.

Optionally also one or more of:
The pipeline is fully automatic.
Cardiac cycles which have inferior quality for robust deformation estimation are automatically discarded and/or a warning is issued.
The pipeline consists of a view recognition step, a segmentation step, a motion estimation step and a regularization step.
View recognition is performed by a convolutional neural network (CNN).
Segmentation is performed by a U-Net type CNN.
Motion estimation is performed by a FlowNet type network.
Automatic discarding of ultrasound data from a cardiac cycle, or issuance of a warning happens when view recognition fails due to view recognition confidence being lower than a threshold.
The view recognition confidence is calculated by the percentage of image frames classified to the same view compared to all frames classified from that cycle.
Automatic discard of ultrasound data from a cardiac cycle happens when segmentation indicates that apex is moving away and towards the probe during the cardiac cycle. (This indicates fore-shortening.)
Automatic discard of ultrasound data from a cardiac cycle happens when regularization is failing.
Processing is applied to several stored cardiac cycles from one patient.
Processing is applied to several stored cardiac cycles from different patients.
Results from several stored cardiac cycles from different patients are used to compare regional strain between patient groups.
A user interface displays a trend graph for regional and/or global values based on processing of current and prior exams for the same patient.
Processing is applied to images from different stress levels and the results are compared—and segments where regional deformation is diverging between stress levels.
A user selects exams to be processed. Later (after processing completed) the user is presented with results showing average regional and global deformation values for all selected exams together with the variance of the results.
Deformation values are used to calculate strain.
Deformation values are used to calculate strain rate.
Automatically, at the end of an examination, the user is presented with a Left ventricle segmental bullseye generated in background based on processing recorded images while the user was completing the exam. Thus, processing occurs automatically on apical B-mode images while the user continues to record colorflow and Doppler images.
Automatically, in background while user continues to record images, processing of already recorded images is performed. If a left ventricle segment falls outside normal deformation value ranges, a warning highlighting this segment and its non-normal value is presented to the user.
Results are presented as bullseye plots.

It should be apparent that the foregoing relates only to the preferred embodiments of the present application and the resultant patent. Numerous changes and modification may be made herein by one of ordinary skill in the art without departing from the general spirit and scope of the invention as defined by the following claims and the equivalents thereof.

We claim:

1. A method for processing echocardiography data in order to enable automatic functional measurements based on cardiac ultrasound images as an input, the method comprising:
(i) classification of the cardiac ultrasound images in order to ensure that relevant images are passed on to the next steps;
(ii) segmentation and semantic partitioning of the left ventricle (LV) myocardium to extract relevant parts of the images;
(iii) regional motion estimates to determine a mapping of displacements in the extracted parts of the image and to output estimated tissue motion vectors for the extracted parts of the image; and
(iv) fusion of measurements via state estimation applied to the tissue motion vectors and thereby incorporating a temporal domain to produce data showing variation of the estimated measurements over time;
wherein at least one of steps (i), (ii) and (iii) is done using a neural network, or a part of a neural network.

2. A method as claimed in claim 1, wherein step (i) uses a first neural network, or a first part of a neural network, step (ii) uses a second neural network, or second part of a neural network, and step (iii) uses a third neural network, or third part of a neural network.

3. A method as claimed in claim 1, wherein step (i) uses a convolutional neural network (CNN) in the form of a feed-forward CNN composed of inception blocks and a dense connectivity pattern.

4. A method as claimed in claim 1, wherein the segmentation at step (ii) uses a U-Net type of CNN classify the LV myocardium with the pixel map of the segmentation being processed and used to define regions of interest (ROI) and the basis of measurement kernels.

5. A method as claimed in claim 3, wherein the segmentation at step (ii) is used for masking to extract the relevant parts of the images and also used for centerline extraction.

6. A method as claimed in claim 5 wherein the contour of the segmentation mask is used to define the endo- and epicardial borders, and the centerline is sampled along the myocard, with the latter being passed to the state estimation at step (iv).

7. A method as claimed in claim 1, wherein the motion estimation at step (iii) uses the neural networks referred to as FlowNets and includes stacking of multiple U-Net architectures with image warping of intermediate motion and propagation of brightness error.

8. A method as claimed in claim 7, wherein step (iii) includes two parallel routes for the motion estimation in order to tackle larger and smaller displacements separately.

9. A method as claimed in claim 8, wherein: larger displacements are predicted by stacking three U-Net architectures, the first which includes explicit correlation of feature maps, while the two succeeding are standard U-Net architectures without custom layers; and wherein for smaller displacements only one U-Net is used, but compared to the networks for the larger displacements, the kernel size and stride of the first layer is reduced.

10. A method as claimed in claim 1, including a further step (v), which comprises calculation of clinical indices.

11. A method as claimed in claim 10, wherein step (v) uses the updated point-velocity components from step (iv) in order to calculate one or more clinical indices selected from: global or regional longitudinal myocardial strain; global or regional longitudinal myocardial strain rate; regional myocardial velocity; and/or regional myocardial displacement.

12. A method as claimed in claim 1, wherein the input cardiac images are cardiac B-mode ultrasound data

13. A method as claimed in claim 1, comprising automatically providing an output comprising regional numeric deformation values for all segments of the left ventricle.

14. A method as claimed in claim 1, wherein cardiac cycles which have inferior quality are automatically discarded during initial processing at step (i).

15. A method as claimed in claim 14 wherein the automatic discarding of ultrasound data occurs: when view recognition fails due to view recognition confidence being lower than a threshold; when segmentation indicates that apex is moving away and towards the probe during the cardiac cycle; and/or when regularization is failing.

16. A method as claimed in claim 1, including outputting information via a user interface that displays a trend graph for regional and/or global values based on processing of as current exam and optionally also prior exams for the same patient.

17. A method as claimed in claim 1, wherein at the end of an exam, the user is automatically presented with a Left ventricle segmental bullseye generated in background based on processing recorded images while the user was completing the exam.

18. A computer programme product containing instructions that, when executed, will configure a computer device to carry out the method of claim 1.

19. A computer device configured to carry out the method of claim 1.

20. An ultrasound imaging system comprising a computer device as claimed in claim 19 for processing ultrasound images obtained via the imaging system.
